(19) United States
(12) Patent Application Publication
(10) Pub. No.: US 2014/0170078 A1
(43) Pub. Date: Jun. 19, 2014

(54) MAGNETIC RESONANCE IMAGING T2 CONTRAST MEDIUM FOR CELL CONTRASTING, AND METHOD FOR MANUFACTURING SAME

(71) Applicants: Taeghwan Hyeon, Gangnam-gu (KR); Nohyun Lee, Seo-gu (KR); Woo Kyung Moon, Gangnam-gu (KR); Seung Hong Choi, Yangcheon-Gu (KR); Hyoungsu Kim, Jongno-Gu (KR)

(72) Inventors: Taeghwan Hyeon, Gangnam-gu (KR); Nohyun Lee, Seo-gu (KR); Woo Kyung Moon, Gangnam-gu (KR); Seung Hong Choi, Yangcheon-Gu (KR); Hyoungsu Kim, Jongno-Gu (KR)

(73) Assignee: SNU R&DB FOUNDATION, Gwanak-Gu (KR)

(21) Appl. No.: 14/123,459

(22) PCT Filed: Jun. 14, 2011
(86) PCT No.: PCT/KR2011/004328

§ 371 Date: Mar. 5, 2014

(51) Int. Cl. A61K 49/18

(52) U.S. Cl. CPC A61K 49/1824
USPC 424/9.322; 424/9.32; 435/29; 427/2.11

(57) ABSTRACT

The invention relates to a magnetic resonance imaging T₂ contrast medium (agent) for cell contrasting, and to a method for manufacturing the same. The magnetic resonance imaging T₂ contrast agent for imaging at cellular level comprises magnetic nanoparticles exhibiting ferrimagnetism at room temperature, has a very high relaxivity, and has an effective uptake into cells. Thus, the T₂ contrast agent may effectively mark various types of cells, and in vitro and in vivo magnetic resonance imaging at the single cell level may he realized.

### TECHNICAL FIELD

The present invention relates to a magnetic resonance imaging T2 contrast agent (medium) and a method for manufacturing the same, more particularly, a magnetic resonance imaging T₂ contrast agent comprising magnetic nanoparticles.

### BACKGROUND AR

Magnetic nanoparticles have attracted much attention for a variety of biomedical applications including MRI, drug delivery, hyperthermia, bioseparation, etc.

Particularly, superparamagnetic iron oxide nanoparticles (SPIOs) such as Feridex, Resovist, etc. have been recently used as a MRI T2 contrast agent since the T2 relaxation time of water adjacent to the nanoparticles is significantly decreased due to high magnetic moment of the nanoparticles. It is possible to increase tiny difference of contrast between tissues by using SPIOs. However, SPIOs generally have relatively low relaxivities since they are synthesized in aqueous media and consequently have poor crystallinity. For ultrasensitive magnetic resonance (MR) imaging, further improvement of r2 relaxivity is strongly desired. Because r2 relaxivity is directly dependent on the magnetic properties of the nanoparticles, there have been several attempts to improve magnetic properties and consequently increase relaxivities by controlling the composition, aggregation, and oxidation state of magnetic nanoparticles.

Recently, active researches in relation to MR imaging have been carried out in order to investigate, in anatomical details, continuous cellular events such as migration of stem cells, immunorejection by macrophages, etc., metastasis of cancer, cell vaccines by using dendritic cells, and progression of arteriosclerosis, etc. In order to spot cell-level events, it is required an ability to image very small number of cells. However, ultrasensitive in vivo tracking of a small number of labeled cells is restricted due to low sensitivity of MR images. In order to improve ultrasensitive cellular MRI capability, there have been various attempts to increase the cellular uptake of nanoparticles, such as conjugation with cell-penetrating peptides (CPPs), encapsulation with dendrimers, and coincubation with transfection agents such as PLL. However, these approaches involve complicated conjugation steps, which lead to cell death by generating transient holes in the cell membrane.

The MR imaging of single cells labeled with micrometer-sized iron oxide nanoparticles (MPIOs) which are micrometer-sized aggregates of superparamagnetic iron oxide nanoparticles has been lately reported and, however, large amount of iron should be internalized in cells in order to obtain the in vivo MR imaging of the single cells since the relaxation of the MPIOs is slightly higher than that of Feridex. Meanwhile, the synthesis of iron oxide nanoparticles of which size and shape is very similar to those of the magnetosomes of magnetotactic bacteria has been reported. Although the magnetosomes are known to have excellent magnetic properties, the capability and applicability as a MRI contrast agent has not yet been reported.

### DISCLOSURE

### Technical Problem

During development of a novel MRI contrast agent at a single-cell level in order to image cellular event, the present inventors accomplished the present invention by confirming that magnetosome-like ferromagnetic nanoparticles are taken up into a cell and, thus, high resolution MR imaging of single cells is possible.

Therefore, the present invention is to provide an MRI T2 contrast agent comprising magnetic nanoparticles which have very high relaxivity and enables to effectively label various cells in order to in vitro and in vivo MR imaging of single cells.

In addition, the present invention is to provide a method for preparing a MRI contrast agent for the cell-level imaging.

### Technical Solution

In one aspect of the present invention, there is provided with an MRI contrast agent for cell contrasting, which comprises magnetic nanoparticles having ferrimagnetism at room temperature, in order to accomplish the above-mentioned technical objectives.

According to one embodiment of the present invention, the magnetic nanoparticles may comprise, for example, magnetite (Fe₃O₄), maghemite (γ-Fe₂O₃), cobalt ferrite (CoFe₂O₄), manganese ferrite (MnFe₂O₄), iron-platinum (Fe—Pt) alloy, cobalt-platinum (Co—Pt) alloy, cobalt (Co) and combinations thereof, but not limited thereto.

According to one embodiment of the present invention, the size of the magnetic nanoparticles may be about 10 nm to about 1,000 nm, preferably about 10 nm to about 200 nm, but not limited thereto.

According to one embodiment of the present invention, the magnetic nanoparticles may comprise magnetite (Fe₃O₄), but not limited thereto.

According to one embodiment of the present invention, the diameter of the nanoparticles comprising the magnetite may be about 10 nm to about 1,000 nm, more preferably about 20 nm to about 200 nm, but not limited thereto.

According to one embodiment of the present invention, the nanoparticle comprising the magnetite may be a cube, a truncated cube, or an octahedron, but not limited thereto.

According to an embodiment of the present invention, the magnetic nanoparticles may be coated with biocompatible materials, but not limited thereto.

According to one embodiment of the present invention, the biocompatible may be selected from the group consisting of, for example, polyvinylalcohol, polylactide, polyglycolide, poly(lactide-co-glycolide), polyanhydride, polyester, polyetherester, polycaprolactone, polyesteramide, polyacrylate, polyurethane, polyvinylflouride, polyvinylimidazole, chlorosulphnate polyolefin, polyethyleneoxide, polyethyleneglycol, dextran, mixtures thereof and copolymer thereof, but not limited thereto.

According to one embodiment of the present invention, the contrast agent may be used for monitoring cell transplantation procedures or transplanted cells in a cell therapy, where the cells to be transplanted are cell therapeutic agents selected from the group consisting of islet cells, dendritic cells, stern cells, immunocytes, and combinations thereof, but not limited thereto.

According to one embodiment of the present invention, the contrast agent may be a nanoparticle coated with the biocompatible material, where biocompatible materials are attached to the outer surfaces of the nanoparticles, but not limited thereto.

In an exemplary embodiment, the biocompatible material may be selected from the group consisting of for example, a protein, RNA, DNA, an antibody and combinations thereof, which attaches specifically to an in vivo target material; an apoptosis-inducing gene or a toxic protein; a fluorescent material; an isotope; a material responsive to a light, an electromagnetic wave, or heat; a pharmacologically active material; and combinations thereof, but not limited thereto.

In another aspect of the present invention, there is provided with a method for producing a magnetic resonance imaging (MRI) T₂ contrast agent for cell contrasting, which comprises: heating a mixture of a metal precursor, a surfactant and a solvent to producing a magnetic nanoparticle which is ferrimagnetic at room temperature; and coating said magnetic nanoparticle with a biocompatible material.

According to one embodiment of the present invention, the diameter of the magnetic nanoparticles comprising the magnetite may be about 10 nm to about 1,000 nm, more preferably about 10 nm to about 200 nm, but not limited thereto.

According to one embodiment of the present invention, the magnetic nanoparticles may comprise magnetite of a size of about 20 nm to about 1,000 nm, more preferably of a size of about 20 nm to about 20 m or 200 nm, but not limited thereto.

According to one embodiment of the present invention, the magnetic nanoparticles comprising magnetites of a size of about 20 nm to about 1,000 nm, but not limited thereto.

According to one embodiment of the present invention, the magnetic nanoparticles comprising the magnetite may be prepared by heating a mixture of an iron precursor, a surfactant and a solvent, but not limited thereto.

According to an embodiment of the present invention, the iron precursor may be selected from the group consisting of, for example, iron (II) nitrate (Fe(NO₃)₂), iron (III) nitrate (Fe(NO₃)₃), iron (II) sulfate (FeSO₄), iron (III) sulfate (Fe₂(SO₄)₃), iron (II) acetylacetonate (Fe(acac)₂), iron (III) acetylacetonate (Fe(acac)₃), iron (II) trifluoroacerylacetonate (Fe(tfac)₂), iron (III) trifluoroacerylacetonate (Fe(tfac)₃), iron (II) acetate (Fe(ac)₂), iron (III) acetate (Fe(ac)₃), iron (II) chloride (FeCl₂), iron (III) chloride (FeCl₃), iron (11) bromide (FeBr₂), iron (III) bromide (FeBr₃), iron (II) iodide (FeI₂), iron (III) iodide (FeI₃), iron perchlorate (Fe(ClO₄)₃), iron sulfamate (Fe(NH₂SO₃)₂), iron (II) stearate ((CH₃(CH₂)₁₆COO)₂Fe), iron (III) stearate ((CH₃(CH₂)₁₆COO)₃Fe), iron (II) oleate ((CH₃(CH₂)₇CHCH(CH₂)₇COO)₂Fe), iron (III) oleate ((CH₃(CH₂)₇CHCH(CH₂)₇COO)₃Fe), iron (II) laurate ((CH₃(CH₂)₁₀COO)₂Fe), iron (III) laurate ((CH₃(CH₂)₁₀COO)₃Fe), penta carbonyl iron (Fe(CO)₅), enneacarbonyldiiron (Fe₂(CO)₉) and combinations thereof, but not limited thereto.

According to one embodiment of the present invention, the surfactant may be selected from the group consisting of, for example, carboxylic acid, alkyl amine, alkyl alcohol, alkyl phosphine and combinations thereof, but not limited thereto.

According to one embodiment of the present invention, the solvent may comprise an organic solvent of which boiling temperature is more than 100° C., and of which molecular weight is 100 to 400, but not limited thereto.

According to one embodiment of the present invention, the solvent may be selected from the group consisting of, for example, hexadecane, hexadecane, octadecane, octadecene, eicosane, eicosene, phenanthrene, pentacene, anthracene, biphenyl, phenyl ether, octyl ether, decyl ether, benzyl ether, squalene and combinations thereof, but not limited thereto.

According to one embodiment of the present invention, the temperature of said heating step may be between 100° C. and the boiling temperature of the solvent, but not limited thereto.

According to one embodiment of the present invention, the heating rate of said heating step may be 0.5′C./min to 50° C./min, but not limited thereto.

According to one embodiment of the present invention, the pressure of said heating step may be 0.5 atm to 10 atm, but not limited thereto.

According to one embodiment of the present invention, the mole ratio of said metal precursor and said surfactant may be 1:0.1 to 1:20, but not limited thereto.

According to one embodiment of the present invention, the mole ratio of said metal precursor and said solvent may be 1:1 to 1:1,000, but not limited thereto.

### Advantageous Effects

Since the contrast agent of the present invention has a very high relaxivity and is effectively taken up into a cell and internalized without additional treatments, it is possible to effectively label various types of cells and to MR image in vitro and in vivo single cells. For example, it is possible to monitor cell transplantation procedures and cell therapies by labeling cells to be transplanted with a cell therapeutic agent such as islet cells.

In addition, it may be difficult to obtain images of single cells by using a clinical 1.5 T MR scanner with a low resolution and low signal-to-noise ratio. However, it is possible to reduce a detection limit of cells when the cells are labeled with the contrast agent of the present invention,

Therefore, it is expected that the imaging of single cell by using the contrast agent of the present invention has a considerable potential for basic biological researches as well as clinical diagnostics and therapies.

### BRIEF DESCRIPTION OF THE DRAWINGS

FIG. 1 shows TEM images of ferromagnetic iron oxide nanoparticles (FIONs) prepared according to one example of the present invention before (FIG. 1a) and after (FIG. 1b) coating with PEG-phospholipid, images of the dispersed and precipitated state of the FIONs (FIG. 1c), a T2-weighted MR image of FION, MPIO and Feridex (FIG. 1d), and plots of r2 value of FION, MPIO and Feridex (FIG. 1e).

FIG. 2 shows an image of MDA-MB-231 breast cancer cells stained with Prussian blue and treated with the FIONs according to one example of the present invention (the cells were coater-stained with NFR (nuclear fast red)) (FIG. 2a), an image of MDA-MB-231 cells suspended after removal of the free FIONs by using Ficoll-Paque (the FIONs are visible as dark spots inside the cells) (FIG. 2b), a TEM image of the FIONs trapped in the vesicle of cells (FIG. 2c), plots of the cytotoxicity of the FIONs to MDA-MB-231 strain (FIG. 2d), and are MR image of T2 relaxation time of the cells labeled with the FIONs (FIG. 2e).

FIG. 3 shows MDA-MB-231 cells (FIG. 3a), hMSC stem cells (FIGS. 3b), and K562 suspension cells (FIG. 3c), into which the FIONs were taken up. These images indicate that various cells may be labeled with the FIONs.

FIG. 4 shows a schematic diagram of a cell phantom for MR imaging of single cells (FIG. 4a), an MR image of four labeled cells sandwiched between two Gelrite (FIG. 4b), a fluorescence image of cells stained with calcein-AM (FIG. 4c), a merged image of corresponding region of FIGS. 4b and 4c (FIG. 4d), and in vivo T2* MR images of brain of control mouse (FIG. 4e: control group) that received no cells and mouse (FIG. 4f: experimental group) that received intracardiac injection of FION-labeled cells.

FIG. 5 shows an image of rat pancreatic islet labeled with the FIONs according to one embodiment of the present invention, and MR images of rat liver transplanted with the pancreatic islets labeled with the FIONs.

FIG. 6 shows a graph of change of magnetism of the magnetite particles with a size thereof.

### BEST MODE FOR CARRYING OUT THE INVENTION

Hereinafter, the present invention is described detail.

In one aspect of the present invention, there is provided with an MRI contrast agent for cell-level contrasting, which comprises magnetic nanoparticles having ferrimagnetism at room temperature.

A ferrimagnetic material is one that has populations of atoms with opposing magnetic moments, as in antiferromagnetism; however, in ferrimagnetic materials, the opposing moments are unequal and a spontaneous magnetization remains. This happens when the populations consist of different materials or ions (such as Fe²⁺ and Fe³⁺).

Examples of magnetic materials exhibiting ferrimagnetic at room temperature include typically magnetite (Fe₃O₄), and also maghemite (γ-Fe₂O₃), cobalt ferrite (CoFe₂O₄), manganese ferrite (MnFe₂O₄), iron-platinum (Fe—Pt) alloy, cobalt-platinum (Co—Pt) alloy, cobalt (Co), etc.

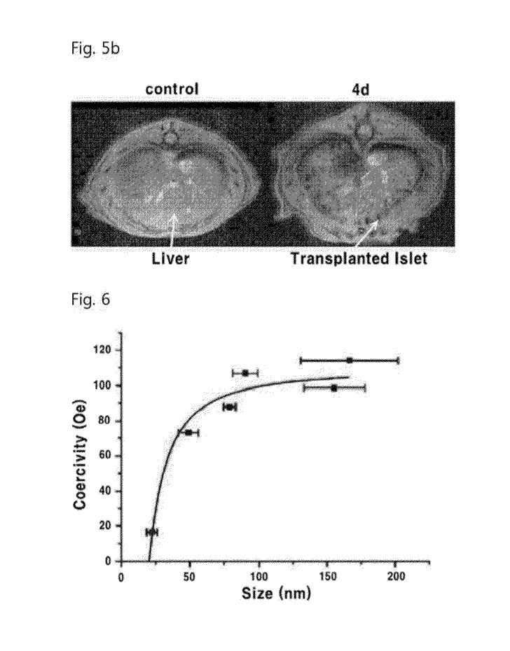

The above-mentioned magnetic materials may be ferrimagnetic according to theft types and sizes. For example, magnetite, maghemite, cobalt ferrite, manganese ferrite, etc. are ferrimagnetic when their sizes are more than about 20 nm. With reference to FIG. 6, the coercivity of the magnetite with a size of less than 20 nm is 0, that is, superparamagnetic, while ferrimagnetic with a size of more than about 20 nm iron-platinum alloy, cobalt-platinum alloy, cobalt, etc. are ferrimagnetic when their sizes are more than about 10 nm. Therefore, the size of the magnetic nanoparticies may be about 10 nm to about 1,000 nm, and the lower limit of the size of the magnetic nanoparticies may be dependent upon the magnetic materials at room temperature. The upper limit of the size of the magnetic nanoparticles may be the range within which the magnetic nanoparticles can be taken up into cells, and may preferably be less than about 200 nm. Since the magnetic nanoparticles with a size of more than about 1,000 nm are difficult to be taken up into cells, they may be inappropriate.

For example, when magnetite is used as magnetic nanoparticles exhibiting ferrimagnetism at room temperature, the size of the magnetite may be about 20 nm to about 1,000 nm. When the size of magnetite nanoparticles are less than about 20 nm, the magnetite nanoparticles are not ferrimagnetic and, thus, are not suitable for the contrast agent of the present invention. Moreover, when the size of magnetite nanoparticles are more than about 1,000 nm, the magnetite nanoparticles cannot be taken up into cells and, thus, are not suitable for the contrast agent of the present invention. The size of the magnetite nanoparticles are preferably about 20 nm to about 200 nm, more preferably about 70 nm to about 80 nm.

The magnetic nanoparticies are preferably uniform-sized, for example, uniform-sized nanoparticles below about 15% of the standard deviation of their average size, preferably below about 10%, more preferably below about 5%.

The shape of the magnetite nanoparticles may be truncated cubic or octahedral, in addition to cubic. For example, cubic ferrimagnetic iron oxide (magnetite) nanoparticles may be used for the contrast agent and, in this case, the ferrimagnetic iron oxide nanocubes (FIONs) may be the same to the magnetosomes of magnetotactic bacteria, in respect of the size and shape.

When the shape of the magnetite nanoparticies are cubic, truncated cubic or octahedral, very high magnetic field exhibits in a certain direction, in contrast with a sphere which is symmetrical to all directions, and thus more degrees of freedom may be given in respect of measuring and utilizing the magnetism of the nanoparticles. In addition, since the surface energy of the iron atoms present at the edge of the cube, truncated cube or octahedral is higher than that of the iron atoms present at the surface of the sphere, there are differences in reactivity.

According to one embodiment of the present invention, the magnetic nanoparticles exhibiting ferrimagnetism at room temperature may be coated with a biocompatible material in order to allow the nanoparticles to be stable in a dispersion state at aqueous environment and to be biocompatible.

The biocompatible material is not toxic in vivo and examples thereof include, for example, polyvinylalcohol, polylactide, polyglycolide, poly(lactide-co-glycolide), polyanhydride, polyester, polyetherester, polycaprolactone, polyesteramide, polyacrylate, polyurethane, polyvinylflouride, polyvinylimidazole, chlorosulphnate polyolefin, polyethyleneoxide, polyethyleneglycol, dextran, mixtures thereof or copolymer thereof. Any biocompatible materials which do not described herein but known to a person skilled in the art may be used for coating materials. The biocompatible material may preferably be polyethyleneglycol, for example, 1,2-disteroyl-sn-glycero-3-phosphoethanolamine-N-[methoxy(polyethyleneglycol)-2000], etc. Therefore, according to one embodiment of the present invention, an MRI T2 contrast agent for cell contrasting may be provided, which is prepared by coating iron oxide nanoparticles having a size of about 20 nm to about 200 nm and being ferrimagnetic at room temperature, with 1,2-disterayl-sn-glycero-3-phosphoethanolamine-N-[methoxy(polyethyleneglycol-2000] as a biocompatible material.

According to one embodiment of the present invention, a bioactive (biologically active) material may be bound to the biocompatible material with which the nanoparticles are coated.

The bioactive material comprises an antibody which recognizes and attaches specifically to a certain antigen, a monoclonal antibody prepared by using the antibody, a variable region or constant region of an antibody, a chimeric antibody of which some or all are modified, a humanized antibody, etc., and also comprises nucleic acids such as DNA or RNA which can complimentarily combine with DNA or RNA that has a certain base sequence, target-specific materials including non-biological chemical compounds which can make a chemical bond to a certain functional group through a hydrogen bonding, etc. at certain conditions, various pharmacologically active materials which treat, prevent or alleviate diseases, toxic active materials which are genes inducing apoptosis or toxic proteins, chemical compounds which react with electromagnetic waves, magnetic fields, electric fields, lights or heats, fluorescent materials and in viva active materials such as isotopes generating radioactive rays. It is possible to introduce various functions such as target-specificity, drug delivery, treatment effects, hyperthermia, etc. to the contrast agent of the present invention through introduction of such bioactive materials.

The bioactive materials to be attached to the contrast agent comprise bioactive materials known in the art, preferably materials that are permeable to cell membranes and do not prevent uptake into cells, for contrasting cells.

In the second aspect of the present invention, there is provided with a method for producing a magnetic resonance imaging (MRI) T₂ contrast agent for cell contrasting, which comprises: heating a mixture of a metal precursor, a surfactant and a solvent to producing a magnetic nanoparticle which is ferrimagnetic at room temperature; and coating said magnetic nanoparticle with a biocompatible material.

In the step for producing a magnetic nanoparticle, the precursor for preparing the magnetic nanoparticles may be selected according to the nanoparticles to be prepared. For example, iron precursor, manganese precursor, cobalt precursor and other precursors may be used as the metal precursor, but not limited thereto.

When the magnetic nanoparticle is magnetite, iron precursors, e.g., iron (II) nitrate (Fe(NO₃)₂), iron (III) nitrate (Fe(NO₃)₃), iron (II) sulfate (FeSO₄), iron (III) sulfate (Fe₂(SO₄)₃), iron (II) acetylacetonate (Fe(acac)₂), iron (III) acetylacetonate (Fe(acac)₃), iron (II) trifluoroacerylacetonate (Fe(tfac)₂), iron (III) trifluoroacerylacetonate (Fe(tfac)₃), iron (II) acetate (Fe(ac)₂), iron (III) acetate (Fe(ac)₃), iron (II) chloride (FeCl₂), iron (III) chloride (FeCl₃), iron (II) bromide (FeBr₂), iron (III) bromide (FeBr₃), iron (II) iodide (FeI₂), iron (III) iodide (FeI₃), iron perchlorate (Fe(ClO₄)₃), iron sulfamate (Fe(NH₂SO₃)₂), iron (II) stearate ((CH(CH₂)₁₆COO)₂Fe), iron (III) stearate ((CH₃(CH₂)₂₆COO)₃Fe), iron (II) oleate ((CH₃(CH₂)₇CHCH(CH₂)₇COO)₂Fe), iron (III) oleate ((CH₃(CH₂)₇CHCH(CH₂)₇COO)₃Fe), iron (II) laurate ((CH₃(CH₂)₁₀COO)₂Fe), iron (III) laurate ((CH₃(CH₂)₁₀COO)₃Fe), pentacarbonyliron (Fe(CO)₅), enneacarbonyldiiron (Fe₂(CO)₉) combinations thereof, etc. may be used as a precursor for producing magnetite.

The surfactant may be, for example, carboxylic acid, alkyl amine, alkyl alcohol, alkyl phosphine and combinations thereof, but not limited thereto.

The carboxylic add may be, for example, octanoic acid, decanoic acid, lauric acid, hexadecanoic acid, oleic acid, stearic acid, benzoic acid, biphenylcarboxylic add, and combinations thereof, but not limited thereto.

The alkyl amine may be, for example, octylamine, trioctylamine, decylamine, dodecylamine, tetradecylamine, hexadecylamine, oleylamine, octadecylamine, tribenzylamine, triphenylamine, and combinations thereof, but not limited thereto.

The alkyl alcohol may be, for example, octyl alcohol, decanol, hexadecanol, hexadecandiol, oleyl alcohol, phenol, and combinations thereof, but not limited thereto.

The alkyl phosphine may be, for example, triphenylphosphine, trioctylphosphine, and combinations thereof, but not limited thereto.

According to one embodiment of the present invention, the solvent may be an organic solvent having a boiling temperature of more than about 100° C. and a molecular weight of about 100 to about 400, for example, hexadecane, hexadecane, octadecane, octadecene, eicosane, eicosene, phenanthrene, pentacene, anthracene, biphenyl, phenyl ether, octyl ether, decyl ether, squalene, and combinations thereof, but not limited thereto.

According to one embodiment of the present invention, the heating temperature of the mixture may be from about 100° C. to the boiling temperature of the solvent used the heating rate may be about 0.5° C./min to about 50° C./min, the pressure of the heating step may be about 0.5 atm to about 10 atm, but not limited thereto.

The mole ratio of the iron precursor and the surfactant may be about 1:0.1 to about 1:20, preferably about 1:1 to about 1:10, and the mole ratio of the iron precursor and the solvent may be about 1:1 to about 1:1,000, preferably about 1:5 to about 1:100.

In the step of producing a magnetic nanoparticle, as the heating time is decreased, more octahedral nanoparticies are produced, and as the heating time is slightly decreased, more truncated cubic nanoparticles are produced. In contrast, when the heating time is increased too much, the surface of the produced nanoparticles may be coarse. For example, the nanoparticles may be produced through the heating time of about 10 min to about 2 hr.

In addition, by adjusting the reaction conditions, the size control of the nanoparticle may be possible and the nanoparticles having an appropriate size allowing for ferrimagnetism may be produced according to types of the nanoparticle. For example, in the case of magnetite nanoparticies, nanoparticles having an average size of about 20 nm to about 1,000 nm, preferably about 20 nm to 200 nm may be produced.

The step of coating the magnetic nanoparticles with a biocompatible material is carried out, subsequent to the step of producing the magnetic nanoparticles.

The biocompatible material is the same to that mentioned above. The coating method may be various methods known to a person in the art in consideration of the biocompatible material, but not limited thereto.

Hereinbelow, examples will be described in order to facilitate the understanding the present invention. However, the examples are given only for illustration of the present invention and not to be limiting the present invention.

### EXAMPLES

### Example 1

### Synthesis of Ferrimmagnetic Iron Oxide Nanocube (FION)

Iron(II) acetylacetonate was added to a mixture composed of oleic acid and benzyl ether. Remaining air was removed by reducing the mixture solution via a vacuum pump. Then, the mixture solution was heated up to the boiling temperature of benzyl ether, while stirring the mixture solution. The mixture solution was maintained at the boiling temperature for 30 minutes and was cooled in air. Then, a mixture of toluene and hexane was added to the mixture solution, thereafter particles were separated by centrifugation. The separated particles were stored in chloroform.

In order to render the magnetic nanoparticles hydrophilic and biocompatible, the synthesized magnetic nanoparticles were mixed with 1,2-distearoyl-sn-glycero-3phosphoethanolamine-N-[methoxy(polyethylene glycol)-20001](mPEG-2000 PE, Avanti Polar Lipids, Inc.) in chloroform. After removing chloroform at 80° C., the nanoparticles were dispersed by adding water. Excessively added 1,2-distearoyl-sn-glycero-3phosphoethanolamine-N-[methoxy(polyethylene glycol)-2000] was removed by centrifugation.

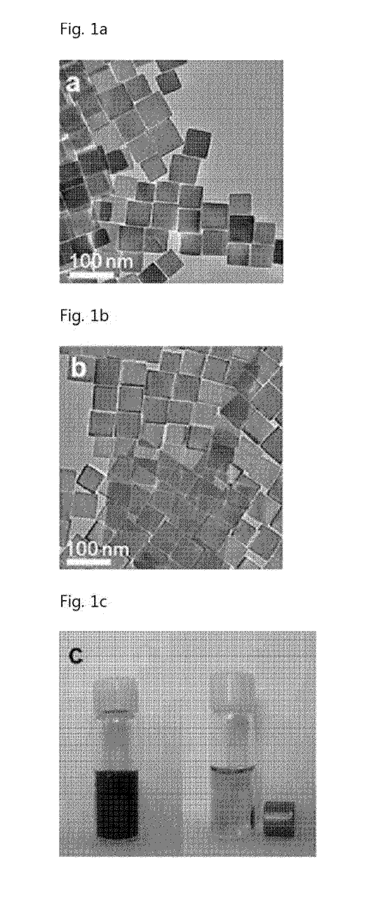

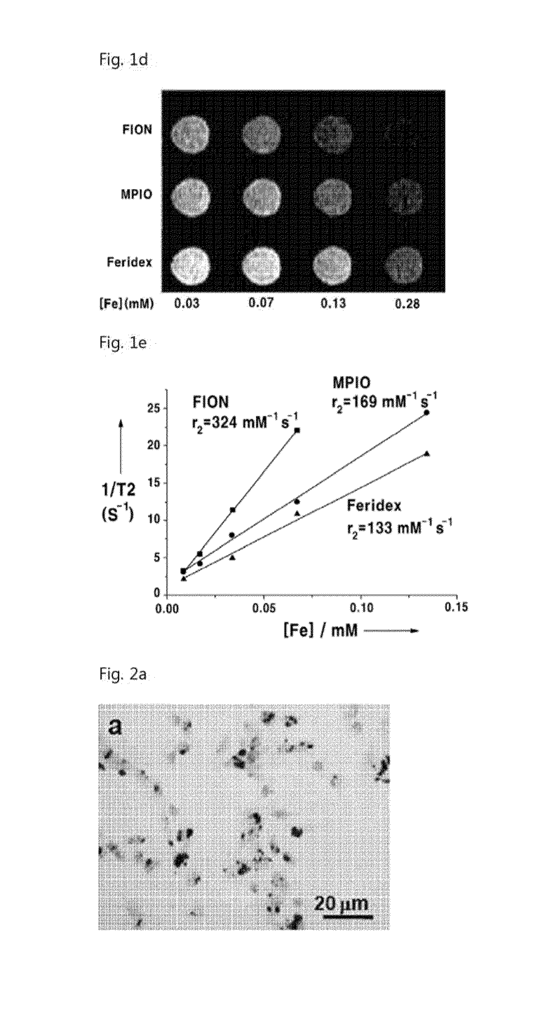

The synthesized FIONs were cubic-shaped and about 20 nm-200 nm, as shown in FIG. 1a. The properties of the synthesized nanoparticles in the organic phase remained, without change of their physical properties during being dispersed in water (FIG. 1b). Also, since the size of the synthesized nanoparticles was bigger than the size where the particles are superparamagnetic, the nanoparticles were ferromagnetic. Thus, the synthesized nanoparticies had strong residual magnetism when there was no external magnetic field. In addition, magnetic interaction between the nanoparticles due to the strong residual magnetism occurred, the nanoparticles were immediately aggregated and, then, precipitated in the solution (FIG. 1c).

### Example 2

### Measurement the Magnetic Resonance Imaging Ability of FION

In order to measure the magnetic resonance imaging ability, various concentrations of the nanoparticles which were synthesized in Example 1, were dispersed in 1% agarose solution. In order to measure T2 relaxation time of the nanoparticles, T2-weighted images were obtained by using a 1.5T magnetic resonance scanner Also, T2 relaxation time was measured from change of signal intensity with change of TE value, by using the Levenberg-Marquardt algorithm.

Since the synthesized nanoparticies have strong contrast effect, they show more distinguished contrast effect in T2-weighted images than the conventional superparamagnetic particles (FIG. 1d). As shown in FIG. 1e, the relaxivity of the FIONs was 2 or 3 times higher than that of Feridex which is currently commercialized.

### Example 3

### Cellular Uptake of the Magnetic Nanoparticles

For cellular uptake of the magnetic nanoparticles synthesized in Example 1, the nanoparticles were cultured for 24 hours together with the prepared cells. The cells were detached from the culture plate by trypsin treatment, in order to remove the nanoparticles which were not taken up. Ficoll-paque was placed in the centrifugation tube, after then the cellular dispersion solution was added carefully on the Ficoll-paque layer, thereby separation between the cellular dispersion solution layer and the Ficoll-paque layer. When the centrifugation was performed at this state, the nanoparticles with high density were settled down under the Ficoll-paque layer, while the cells with low density were floated on the Ficoll-paque layer.

In order to stain the intracellular magnetic nanoparticles, the cells from which unabsorbed nanoparticles were removed were cultured in an 8-well chamber slide and were fixed with 4% paraformaldehyde. The iron ions of the nanoparticles in the fixed cells were stained by adding a mixture composed of potassium ferrocyanide and hydrochloric acid, and then the cells were stained by adding a Nuclear Fast Red solution.

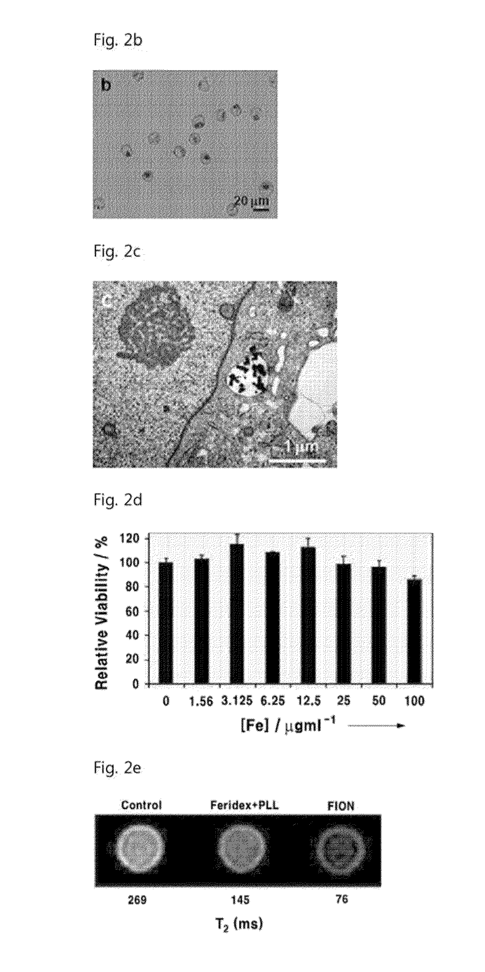

As shown in Prussian Blue staining image of FIG. 2a, most of the cells contain the nanoparticles, and the nanoparticles which were not taken up were not observed after separation by using the Ficoll-paque. Since the stained nanoparticles were taken up within the cells, not attached on the surface of the cells, the nanoparticles were observed inside the cells even after detaching the cells by using trypsin (FIG. 2b). Since the uptakes of the nanoparticles into the cells are occurred via endocytosis, the nanoparticles were detached in endosomes in the cells (FIG. 2c).

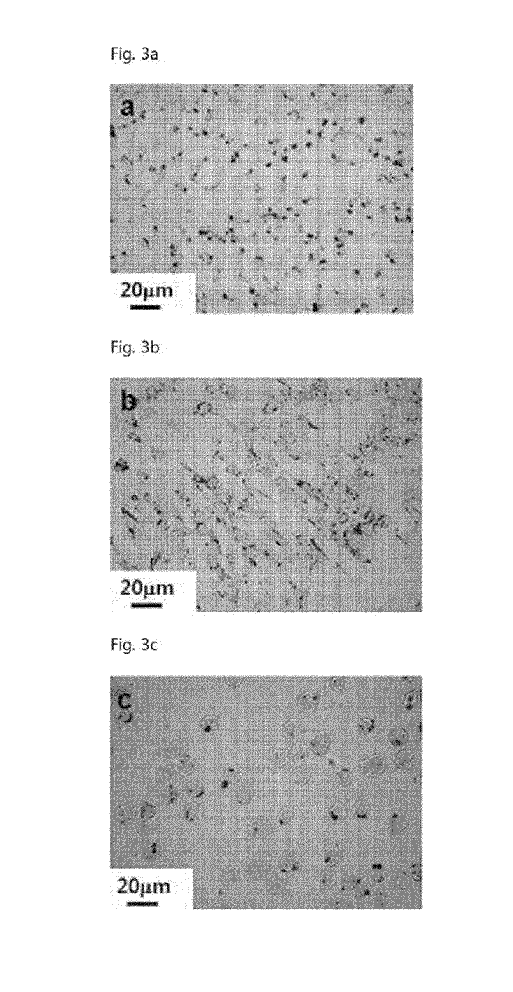

The cellular uptake of the FIONs was observed in various cells. As shown in FIG. 3, the uptake of nanoparticles were observed in MDA-MB-231 (a cancer cell), hMSC (a stem cell), and K562 (a suspension cell). The uptake of the FIONs into the cells due to the endocytosis occurred after sedimentation of the nanoparticles on the cell surface, and the amount of the nanoparticles taken up relatively decreased since the suspension cells were hard to contact nanoparticles.

The cells which taken up the nanoparticles were dispersed in agarose, and then MR imaging was carried out at 1.5T. When the cells were labeled with the FIONs, very strong contrasting effect was shown. The contrasting effect of FIONs was stronger than that of the currently commercialized Feridex, even when the cells were treated with Feridex and poly-1-lysine simultaneously where Feridex is taken up the most into cells (FIG. 2e).

### Example 4

### Evaluation of Toxicity of the Magnetic Nanoparticles

The toxicity of the magnetic nanoparticles were evaluated by MTT (3(4,5-Dimethylthiazol-2-yl)-2,5-Diphenyltetrazolium Bromide) assay. In order to carry out the MTT assay, cells were cultured in a 96-well and, then, 1 μg Fe/ml to 100 μg Fe/ml of the nanoparticles were added. After 24 hours, the culture medium was removed and, then, 0.1 mg/ml of MTT solution was added and cultured for 1 hour, followed by removing the MTT solution. The precipitated violet reduced crystals were dissolved by adding DMSO and the absorbance was measured at 560 nm. As a result, no appreciable toxicity was observed up to 100 μg Fe/ml of the nanoparticles.

### Example 5

### MR Imaging of Single Cells

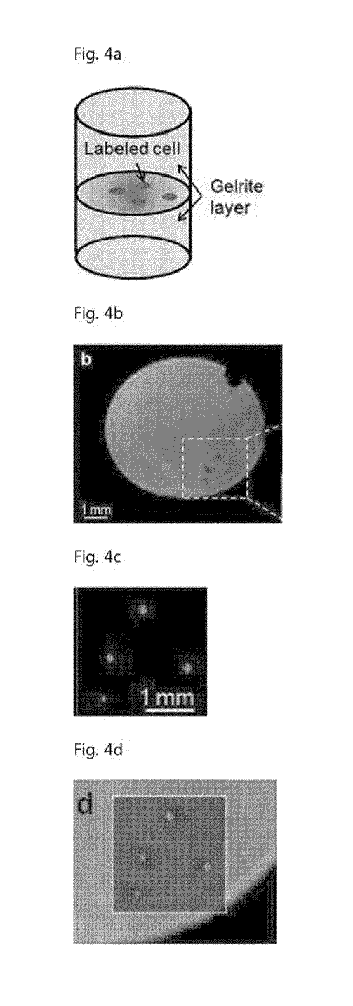

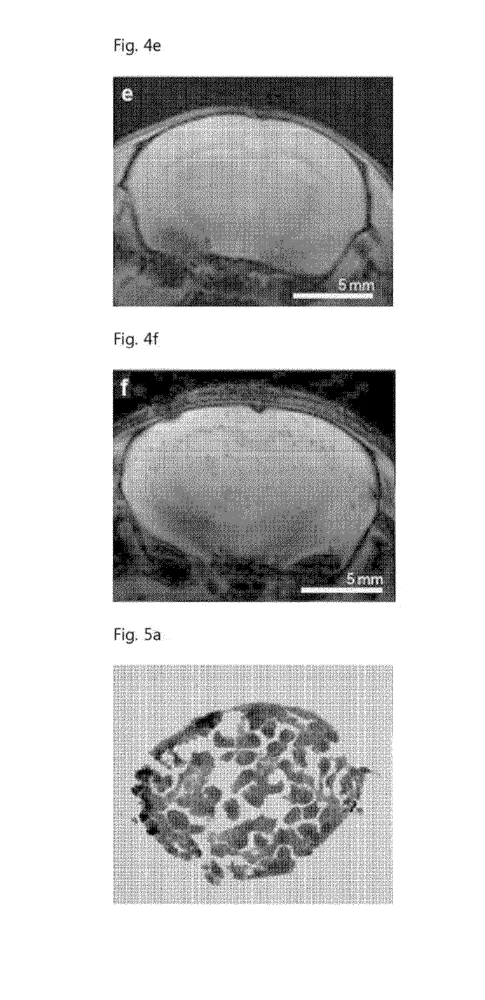

As described in Example 3, cells were cultured with the magnetic nanoparticies. After removing the nanoparticles which were not taken up, the cells were fluorescently labeled with Calcein-AM. After cutting a 96-well ELISA plate into 2×2 wells, several cells were carefully placed between Gelrite layers (FIG. 4a). MR imaging were performed by using 9.4 T magnetic resonance imaging and the conditions of the MR imaging were as follows: flip angle=90 deg, TR=5000 ms, TE=13.1 ms, NEX=4, FOV=2.5 cm×2.5 cm, matrix=256×256, thickness=0.5 mm.

As shown in FIG. 4b, the cells labeled with the FIONs were shown as dark spots in the MR images. It could be understood that the location of the dark spots coincided with that of the cells obtained from the fluorescence image (FIGS. 4c and 4d).

### Example 6

### In Vivo MR Imaging of Single Cells

As described in Example 3, cells were prepared by culturing with the magnetic nanoparticles. The thus prepared cells were added to a serum-free DMEM solution and were injected to left ventricle of a rat. MR imaging was performed by using 9.4 T MRI, 1 hr after the injection. The MR imaging conditions were as follows: flip angle=90 deg, TR=5000 ms, TE=7.6 ms; NEX=1, FOV=2.0 cm×2.0 cm, matrix=256×256, thickness=1 mm.

After the injection of the cells, the brain of the rat was observed by using an MRI and the cells were observed as dark spots (FIGS. 4e and 4f). In order to observe directly the distribution of the cells in the brain, the brain was extirpated and stained with Prussian Blue. Then, the cells were observed in the stained brain.

### Example 7

### Labeling of Pancreatic Islets and In Vivo MR Imaging

Pancreatic islets of the rat was cultured in a FION solution having a concentration of 25 μg/ml for 24 hr and the takeup of the nanoparticles was observed by staining with Prussian blue (FIG. 5a). The cultured pancreatic islets were transplanted to a liver through a portal vein and MR imaging was carried out. FIG. 5b shows a control group and the rat transplanted with the pancreatic islets. The transplanted pancreatic islets were shown as dark spots in the MR image.

The preferred examples of the present invention were described above. However, it can be understood that a person skilled in the art can modify or alter the present invention within the scope of the invention.

1. A magnetic resonance imaging (MRI) T₂ contrast agent for cell contrasting, comprising a magnetic nanoparticle which is ferrimagnetic at room temperature.

2. The MRI T₂ contrast agent of claim 1, wherein said magnetic nanoparticle is selected from the group consisting of magnetite (Fc₃O₄), maghemite (γ-Fe₂O₃), cobalt ferrite (CoFe₂O₄), manganese ferrite (MnFe₂O₄), iron-platinum (Fe—Pt) alloy, cobalt-platinum (Co—Pt) alloy, cobalt (Co) and combinations thereof.

3. The MRI T₂ contrast agent of claim 1, wherein the size of said magnetic nanoparticle is 10 nm to 1,000 nm.

4. The MRI T₂ contrast agent of claim 1, wherein the size of said magnetic nanoparticle is 10 nm to 200 nm.

5. The MRI T₂ contrast agent of claim 1, wherein said magnetic nanoparticle comprises magnetite (Fe₃O₄).

6. The MRI T₂ contrast agent of claim 5, wherein the size of said magnetic nanoparticle comprising magnetite (Fe₃O₄) is 20 nm to 1,000 nm.

7. The MRI T₂ contrast agent of claim 5, wherein the size of said magnetic nanoparticle comprising magnetite (Fe₂O₄) is 20 nm to 200 nm.

8. The MRI T₂ contrast agent of claim 5, wherein said magnetic nanoparticle comprising magnetite (Fe₃O₄) is cubic, truncated-cubic, or octahedral.

9. The MRI T₂ contrast agent of claim 5, wherein said magnetic nanoparticle comprising magnetite (Fe₃O₄) is cubic.

10. The MRI T₂ contrast agent of any one of claims 1 to 9, wherein said magnetic nanoparticle is coated with a biocompatible material.

11. The MRI T₂ contrast agent of claim 10, wherein said biocompatible material is selected from the group consisting of polyvinylalcohol, polylactide, polyglycolide, poly(lactide-co-glycolide), polyanhydride, polyester, polyetherster polycaprolactone, polyesteramide, polyacrylate, polyurethane, polyvinylflouride, polyvinylimidazole chlorosulphnate polyolefin, polyethyleneoxide, polyethyleneglycol, dextran mixtures thereof and copolymer thereof.

12. The MRI T₂ contrast agent of claim 10, wherein said biocompatible material comprises polyethyleneglycol.

13. The MRI T₂ contrast agent of claim 10 for use in monitoring cell transplantation or transplanted cell in a cell therapy.

14. The MRI T₂ contrast agent of claim 13, wherein said transplanted cell is selected from the group consisting of an islet cell, a dendritic cell, a stem cell, an immune cell and combinations thereof.

15. The MRI T₂ contrast agent of claim 10, wherein a bioactive material is attached to the surface of said magnetic nanoparticle coated with biocompatible material.

16. The MRI T₂ contrast agent of claim 10, wherein said bioactive material is selected from the group consisting of a protein, RNA, DNA, an antibody and combinations thereof, which attaches specifically to an in vivo target material; an apoptosis-inducing gene or a toxic protein: a fluorescent material: an isotope: a material responsive to a light, an electromagnetic wave, or heat: a pharmacologically active material; and combinations thereof.

17. A method for producing a magnetic resonance imaging (MRI) T₂ contrast agent for cell contrasting, which comprises:
heating a mixture of a metal precursor, a surfactant and a solvent to producing a magnetic nanoparticle which is fern magnetic at room temperature; and
coating said magnetic nanoparticle with as biocompatible material.

18. The method of claim 17, wherein the size of said magnetic nanoparticle which comprises magnetite, is 20 nm to 1,000 nm.

19. The method of claim 18, wherein said nanoparticle comprising magnetite is produced by beating a mixture of an iron precursor, a surfactant and a solvent.

20. The method of claim 19, wherein said iron precursor is selected from the group consisting of iron (II) nitrate (Fe(NO₃)₂), iron (III) nitrate (Fe(NO₃)₃), iron (II) sulfate (FeSO₄), iron (III) sulfate (Fe₂(SO₄)₃), iron (II) acetylacetonate (Fe(acac)₂), iron (III) acetylacetonate (Fe(acac)₃), iron (II) trifluoroacetylacetonate (Fe(tfac)₂), iron (III) trifluoroacerylacetonate (Fe(tfac)₃), iron (II) acetate (Fe(ac)₂), iron (III) acetate (Fe(ac)₃), iron (II) chloride (FeCl₂), iron (III) chloride (FeCl₃), iron (II) bromide (FeBr₂), iron (III) bromide (FeBr₃), iron (II) iodide (FeI₂), iron (III) iodide (FeI₃), iron perchlorate (Fe(ClO₄)₃), iron sulfamate (Fe(NH₂SO₃)₂), iron (II) stearate ((CH₃(CH₂)₁₆COO)₂Fe), iron (III) stearate ((CH₃(CH₂)₁₆COO)₃Fe), iron (II) oleate ((CH₃(CH₂)₇CHCH(CH₂)₇COO)₂Fe), iron (III) oleate ((CH₃(CH₂)₇CHCH(CH₂)₇COO)₃Fe), iron (II) laurate ((CH₃(CH₂)₁₀COO)₂Fe), iron (III) laurate ((CH₃(CH₂)₁₀COO)₃Fe), pentacarbonyliron (Fe(CO)₅), enneacarbonyldiiron (Fe₂(CO)₉) and combinations thereof.

21. The method of claim 17, wherein said surfactant is selected from the group consisting of carboxylic acid, alkyl amine, alkyl alcohol, alkyl phosphine and combinations thereof.

22. The method of claim 17, wherein said solvent: comprises an organic solvent of which boiling temperature is more than 100° C., and of which molecular weight is 100 to 400.

23. The method of claim 17, wherein said solvent is selected from the group consisting of hexadecane, hexadecane, octadecane, octadecene, eicosane, eicosene, phenanthrene, pentacene, anthracene, biphenyl, phenyl ether, octyl ether, decyl ether, benzyl ether, squalene and combinations thereof.

24. The method of claim 17, wherein the temperature of said heating step is between 100° C. and the boiling temperature of said solvent.

25. The method of claim 17, wherein the heating rate of said heating step is 0.5° C/min to 50° C./min.

26. The method of claim 17, wherein the pressure of said beating step is 0.5 atm to 10 atm.

27. The method of claim 17, wherein the mole ratio of said metal precursor and said surfactant is 1:0.1 to 1:20.

28. The method of claim 17, wherein the mole ratio of said metal precursor and said solvent is 1:1 to 1:1,000.
